(19) United States
(12) Patent Application Publication
(10) Pub. No.: US 2006/0149128 A1
(43) Pub. Date: Jul. 6, 2006

(54) Method and devices for lubricating an endoscopic surgical tool

(75) Inventor: Yakov Baror, Haifa (IL)

Correspondence Address: BRUCE E. LILLING;LILLING & LILLING P.C., P.O. BOX 560, GOLDEN BRIDGE, NY, 10526, US

(73) Assignee: Sightline Technologies Ltd., Haifa (IL)

(21) Appl. No.: 11/300,814

(22) Filed: Dec. 14, 2005

Related U.S. Application Data
(60) Provisional application No. 60/641,639, filed on Jan. 5, 2005.

(51) Int. Cl. A61B 1/00

(52) U.S. Cl. 600/104; 600/154

(57) ABSTRACT

The device for lubricating an endoscopic tool comprises a housing accommodating therein a retaining bushing for receiving the surgical tool thereinto. The bushing is provided with a longitudinal through going bore extending between an entry opening and an exit opening. The bore is filled up with a lubricating material and said entry and said exit opening are covered by a respective entry and exit closure that is piercable by the endoscopic tool during insertion thereof in the bushing through the entry opening and protracting thereof distally along the bore.

### FIELD OF THE INVENTION

The present invention relates generally to endoscopy, and specifically is intended for use with an endoscopic tool which during endoscopic procedures is periodically inserted and withdrawn from a working channel of an endoscope. More particularly the invention refers to a solution for improving sliding ability of the endoscopic tool when it displaces within a working channel of an endooscope.

### BACKGROUND OF THE INVENTION

Endoscopes commonly have working channels, running from a proximal port located at the operating handle to a distal end of the endoscope. When the distal end of the endoscope is inserted into the body, the working channel may be used, inter alia, to pass a surgical instrument through the channel to the distal end of the endoscope in order to perform a surgical procedure, e.g. a biopsy or any other procedure. It is well known that insertion and withdrawal of the surgical tool is associated with friction inside the working channel and therefore lubrication of either the inserted instrument or the channel becomes desirable. It should be mentioned that the problem of friction arises not only during passage of the surgical instrument, but also during displacing the shaft of the endoscope within the body passage. It is obvious that an improved sliding property of a scope's shaft against the body passage, e.g. colonoscope against the colon, or of a surgical tool against the working channel, is highly desirable.

Attempts for improving sliding property of the scope's shaft or the surgical tool are known.

For example, Axon (WO 97/43941) discloses a removable cover for an endoscope shaft. The cover comprises a sleeve of elastic material having a low friction external surface configured so that the frictional resistance to forward displacement of the shaft is less than frictional resistance to reverse movement. Unfortunately this solution is complicate since it necessitates providing a dedicated cover, which should be put on the shaft before commencing the endoscopic procedure. Furthermore this solution would not be suitable for lubricating the surgical tool, since it limits the allowable outside diameter of the tool's shaft.

In Chang (US Publication 2003/0220544) there is disclosed a method of delivering lubricant for endoscopic procedures. According to this method, the sliding property of the endoscope within the body passage can be improved if an aqueous lubricant is injected into the suction/instrument channel of the endosocpe to lubricate the area of the body passage around the distal end of the endoscope. It is mentioned that by this method the sliding property of the medical instruments, passing the instrument channel, could be also improved, if the aqueous lubricant is injected into the channel. The disadvantage of this method is associated with the fact that the lubricant exits from the working channel and comes in physical contact with the body tissues, which potentially might be harmful for a patient. The lubricant exits the working channel in especially large amounts when a medical instrument is inserted therein, since the instrument works as a piston, pushing the lubricant forward and forcing it to escape the channel.

It is also stated in the above reference, that at present the instruments intended for passing through the working channel are lubricated by applying a drop of silicone lubricant or other lubricant onto the instrument. However as with the lubricant applied to the endoscope itself, the lubricant tends to be rubbed off as the instrument moves through the channel, particularly at the distal end of the instrument.

In practice the lubricant is applied on the shaft of the endoscopic tool by spraying. This procedure is not environmentally friendly and does not allow distributing the lubricant evenly.

There also exist additional references, mentioning placement of a liquid or solid lubricant around the shaft of the endoscope or within the working channel, e.g. Singh (U.S. Pat. No. 6,599,237) and Abe (US Publication 2003/0023143).

### SUMMARY OF THE INVENTION

Embodiments of the present invention provide a method and devices for use in lubricating an endoscopic surgical tool when it is being displaced along the working channel of an endoscope. Such lubricating improves the sliding ability of the surgical tool, facilitates displacement of the surgical tool within the working channel and renders the surgical procedure more convenient and efficient.

Embodiments of the present invention are particularly (although not exclusively) suited for use with the surgical tools intended for endoscopes, which are provided with sheathing systems for covering the outer surface of an endoscope. Such systems are described for example in Eizenfeld (WO 2004/016299—PCT/IL2003/00661), which is incorporated herein by reference. These systems employ endoscope sheaths which cover the endoscope while the endoscope is inside the patient's body, so that the endoscope remains clean. The sheath is removed at the end of the endoscopic procedure when the endoscope is outside the patient's body and the endoscope is reused.

It should be kept in mind, however, that the present invention is suitable for lubricating surgical tools intended for use in any other endoscopes, including endoscopes, which are not provided with any sheathing system or those which are provided with other sheathing systems known in the art. An example of such a sheathing system is described in Aizenfeld (WO 2005/023100—PCT/IL04/000710), which is incorporated herein by reference. This sheathing system typically include disposable working channels or a disposable internal sleeve, known also as multilumen tubing, that fits inside the working channel of the endoscope and protects it from contamination.

The present invention seeks to provide a solution, which is free from the above mentioned disadvantages of the known in the art lubricating methods and devices, but which retains their advantages.

The main object of the present invention is providing a new solution for lubrication of surgical tools, which is simple, inexpensive, environmentally friendly, efficient and safe for a patient.

The present invention refers to a method for lubricating and to a device for its implementation. Embodiments of this device include either a stand alone disposable device suitable for lubricating various endoscopic surgical tools, like biopsy forceps, polypectomy snares, surgical baskets etc. or a ready-for-use combination of an endoscopic tool fitted with a lubricating device. In the further description, all possible tools, which are suitable for use with an endoscope, are referred-to either as surgical tools or as endoscopic tools.

By virtue of the present invention it is possible to lubricate the tool outside the body, before its insertion in the working channel. Optionally, the tool may subsequently be protracted distally out of the working channel and afterwards retracted back into the working channel, as might be required by the surgical procedure. During protracting the tool, a very homogeneous layer of lubricant is applied to the tool shaft, while, during retracting the tool backward, this layer is rubbed off. At the end of the surgical procedure, the tool is fully withdrawn from the working channel and disposed off together with the disposable lubricating device.

For a better understanding of the present invention as well of its benefits and advantages, reference will now be made to the following description of its embodiments taken in combination with the accompanying drawings.

### BRIEF DESCRIPTION OF THE DRAWINGS

FIG. 1 is a cross-sectional view of a disposable lubricating device in accordance with an embodiment of the present invention;

FIG. 2 is a cross-sectional view depicting the lubricating device shown in FIG. 1 and a surgical tool being lubricated;

FIG. 3 is a cross-sectional view depicting another embodiment of the lubricating device of the invention.

FIG. 4 is a cross-sectional view of a further embodiment of the invention, which refers to a combination of a surgical tool fitted with the lubricating device;

FIG. 5 is a cross-sectional view, showing the combination shown in FIG. 4 in use; and

FIG. 6 is a cross-sectional view, depicting an embodiment of the present invention when it is employed with a sheathing assembly for covering the surgical tool.

### DETAILED DESCRIPTION OF THE INVENTION

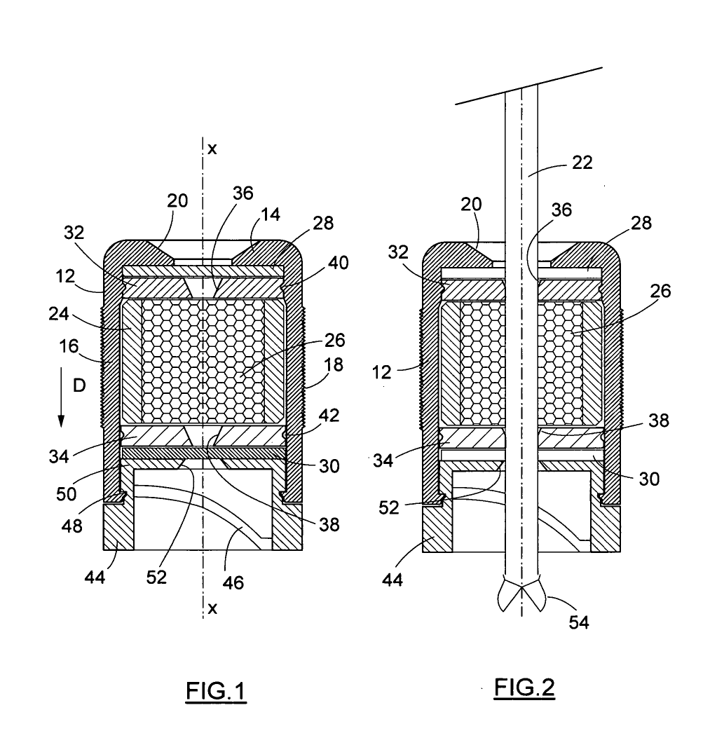

FIG. 1 is a schematic, pictorial illustration of an embodiment of the present invention, which is designated by reference numeral 10. In this embodiment the present invention refers to a lubricating device intended for use as a stand-alone disposable item. The lubricating device is provided with a cup-like external housing 12 defined by a bottom portion 14 and a lateral cylindrical portion 16. A roughed region 18 is provided on the outwardly facing cylindrical surface of the lateral portion. The roughed region enables a reliable grip and convenient manipulation with the lubricating device during its use. In the bottom portion of the housing a conical entry opening 20 is provided for inserting a surgical tool 22. Accommodated within the housing a lubricating assembly is provided. The lubricating assembly consists of a retaining bushing 24 for storing a lubricant 26, first and second cover foils 28, 30 and first and second sealing rings 32, 34. The interior of the retaining bushing is filled up with the lubricant, while cover foils 28, 30 seal opposite ends of the bushing and prevent the lubricant from escaping from the bushing. Each sealing ring is situated between a respective cover foil and a respective bushing end. The sealing rings are provided with corresponding conical apertures 36, 38, which are dimensioned and configured to fit around the shaft of the surgical tool when it displaces along a longitudinal axis X-X of the lubricating device. Each aperture is shaped to allow insertion of the tool and protracting the shaft with little resistance only in a forward (distal) direction while a tight seal is ensured between the narrow sides of the apertures and the shaft during retracting the tool in a backward (proximal) direction. In FIG. 1 an arrow D designates the distal direction. Accordingly, the bottom end of the housing will be referred to further as a proximal end and the opposite open end of the housing will be referred to as a distal end.

Each sealing ring is retained in place by snapping engagement between a respective annular protrusion 40, 42 made on the inwardly facing surface of the housing and an annular recess made on the lateral surface of the respective sealing ring.

Coupled with the distal end of the housing an adaptor 44 is provided. By virtue of this provision the lubricating device can be easily secured on a proximal port of an endoscope to allow lubricating the tool before it is inserted in the working channel. For the sake of brevity neither the port nor the endoscope or working channel are depicted, however they can be seen in our co-pending patent application U.S. Ser. No. 60/641,638 “Improved Surgical Tool For Endoscope,” which is incorporated herein by reference. The adaptor can be secured on the port by a bayonet connection, that includes a helical path 46 made on the inner wall of the adaptor and a corresponding protrusion, which should protrude from the port.

The adaptor is detachably coupled to the housing by snapping engagement between an annular protrusion 48 provided on the distal end of the housing and a corresponding recess made in the adaptor.

Lubricant 26 is retained within bushing 24 since its opposite ends are sealed by cover foils 28, 30. Cover foil 28 is held in place between the bottom portion of the housing and the sealing ring 40, while cover foil 30 is held in place between sealing ring 42 and a lower portion 50 of the adaptor. It is seen also that in the lower portion of the adaptor a conical aperture 52 is provided, which is co-axial with aperture 38 of the sealing ring 34, but it widens in the opposite direction. It should be appreciated that, when adaptor 44 is secured on the proximal port, aperture 52 is brought in alignment with the working channel.

In practice, external housing 12, retaining bushing 24 and adaptor 44 are rigid components, which are manufactured by injection molding or by machining from suitable plastic material, e.g. PVC, polycarbonate, polystyrene, nylon 66, etc.

Cover foils 28, 30 are flat discs manufactured from thin polyamide or polyethylene foil. The cover foils have a thickness that is sufficient to prevent escaping of the lubricant from the bushing. At the same time this thickness allows easy piercing of the foils by a working element 54 of the surgical tool, when it is inserted in the lubricating device through entry opening 20 and then is protracted in the distal direction. In practice, the preferred thickness of the cover foils lies between 5-10 microns.

The working element shown in FIG. 2 is a biopsy forceps, which are operable to take a tissue sample within the patient's body. It should be appreciated, however, that this is only an example and the working element can be of any kind as might be required for the endoscopic procedure, e.g. a polypectomy snare, a basket, etc.

Sealing rings 32, 34 are manufactured from silicon rubber or from any other suitable material, whose resiliency ensures snug fitting of conical apertures 36, 38 around the shaft of the surgical tool. The resiliency of the rings should also be sufficient for reliable sealing when the tool is protracted in the distal direction. On the other hand, the resiliency should provide rubbing off the lubricant from the shaft when the tool is being retracted in the proximal direction.

As suitable lubricant one can use any commercially available organic or inorganic lubricant. Among suitable lubricants one can mention paraffins, silicon oils, glycerin, water solutions of resins, water solutions of polyethylene oxide, vegetable oils, etc.

Attention is drawn to FIG. 2 that schematically shows how surgical tool 22 is lubricated within the lubricating device of the invention. It is not presented, but should be appreciated that the lubricating device is secured on the proximal port of an endoscope.

It is seen that working element 54 of the surgical tool protrudes outside from the lubricating device through entry opening 20 after the tool has been forcibly protracted in the distal direction. For doing this, an operator should have grasped the lubricating device by his/her one hand, while pushing the tool in the distal direction by his/her second hand. On its way distally out the tool bursts cover foil 28, passes retaining bushing, bursts cover foil 30 and exits from the adaptor through aperture 52. It can be readily appreciated that during its displacement along the lubricating device, the tool always remains in contact with the lubricant, so, when the tool exits from the lubricating device, it is covered by an even, homogeneous layer of lubricant. This layer facilitates further advancement of the tool within the working channel or a body lumen.

At the same time since the narrowing apertures 36, 38 tightly fit around the shaft the lubricant is prevented from leaking out.

When the tool is retracted in the proximal direction it easily passes aperture 52, which narrows in the proximal direction. However, when the tool passes resilient sealing rings 34, 32 it meets the narrowing sides of conical apertures 38, 36, which tightly fit around the shaft and rub the lubricant off the tool.

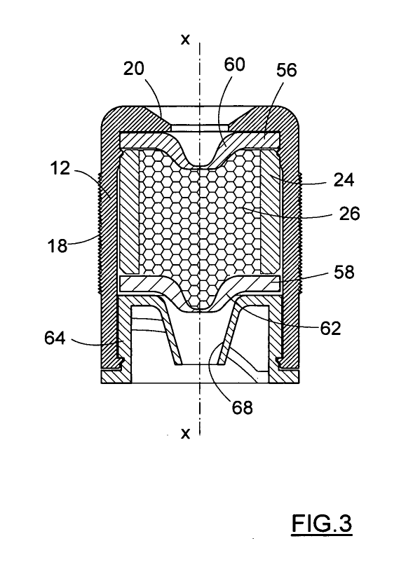

Now with reference to FIG. 3, a further embodiment of the lubricating device will be explained. Some of the components of this embodiment comprises are common with the previous embodiment and accordingly they are designated by the same reference numerals. Among those components are external housing 12 with roughed region 18, entry opening 20 and retaining bushing 24 filled up with lubricant 26.

However, in this embodiment are employed dedicated resilient foils 56, 58 which not only close the retaining bushing, but also function as the sealing rings mentioned in connection with the previous embodiment. This is achieved by providing the foils with a central conical region 60, 62, whose thickness gradually diminishes from a larger size (distant from the axis X-X) to a smaller size (near to the axis X-X). By virtue of this provision, the conical regions tightly fit around the tool after they are burst and either seal or rub the lubricant off from the tool depending whether the tool is protracted or retracted.

An adaptor 64 is provided, whose lower portion 66 is made with a resilient conical aperture 68. The aperture snugly fits around the tool and provides some guiding during displacement of the surgical tool within the working channel.

The previously described embodiments refer to lubricating devices intended for use as a sole, stand alone item. Before use, the lubricating device has to be secured on the proximal port and then the surgical tool has to be inserted in the lubricating device through the entry opening. Upon completing the surgical procedure, the tool is retracted from the proximal port, the lubricating device is detached from the proximal port and is disposed off together with the surgical tool.

Now still further embodiment of the invention will be described. This embodiment refers to an integral item, which is a ready for use combination of the lubricating device with a surgical tool already inserted within it.

In use, such a combination is more convenient since preliminary entering of the tool in the lubricating device is not required any more.

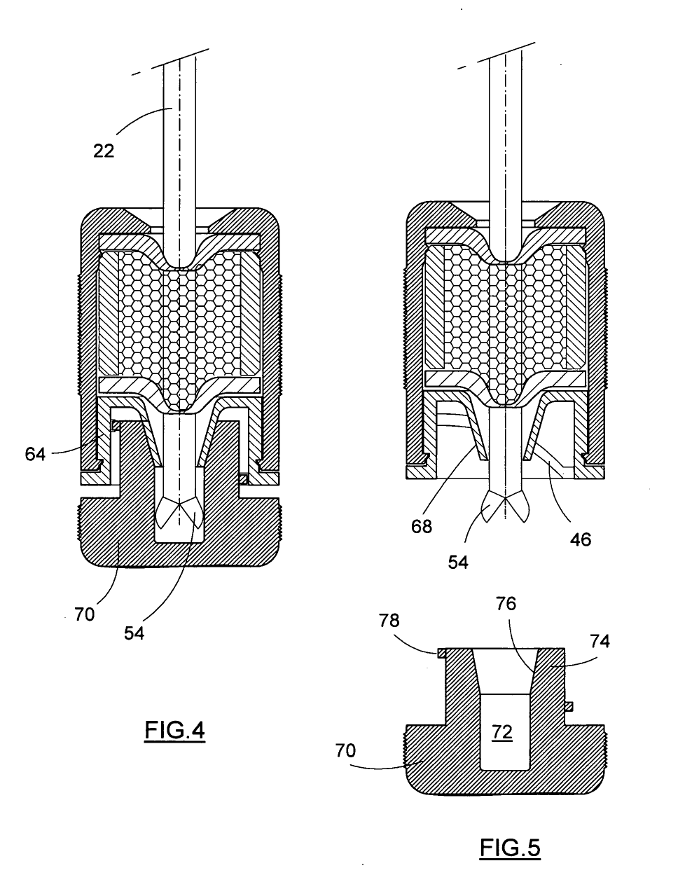

Referring to FIG. 4 an embodiment of such a combination is shown, which employs the same lubricating device and the same surgical tool as in the previous embodiments.

It is advantageous if a cover 70 is detachably connected to adaptor 64 to protect the working end 54 of the surgical tool from contamination or possible damage when the combination is not in use.

Attention is drawn now to FIG. 5, showing a situation when the cover 70 is detached from the adaptor. The cover can be made from the same plastic material as retaining bushing 24. The cover has a lower portion 74 with a receiving recess 72 for accommodating therein the working element of the tool. The recess comprises a closed end and an open end 76, which widens in the proximal direction. The open end mates with the conical aperture 68 and thus some lateral pressure can be exerted on the tool shaft when the cover is put on the adaptor. By virtue of this provision, the positioning of the tool within the retaining bushing is reliably fixed.

The cover can be coupled with the adaptor by any suitable connection, which is known in the art and which allows detachment. In the embodiment shown in FIGS. 4, 5, a bayonet connection is employed, which comprises helical path 46 provided in the adaptor and corresponding protrusion 78 made on the lower portion of the cover.

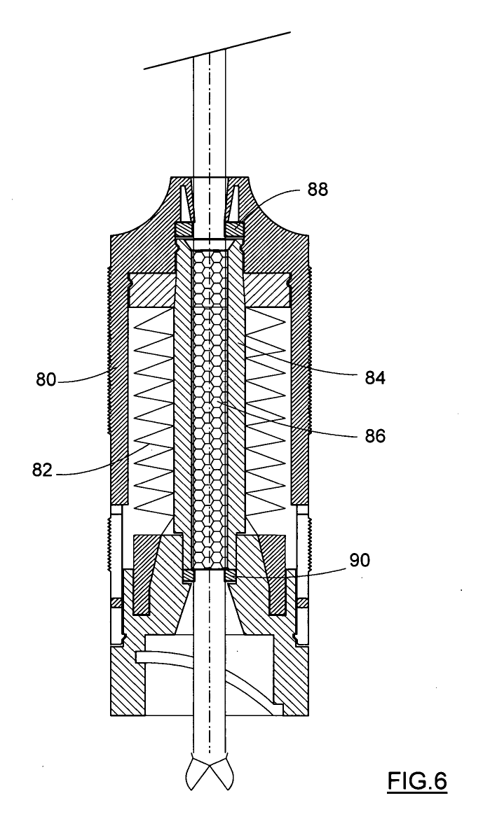

Now with reference to FIG. 6 still a further embodiment of the lubricating device is shown. In this embodiment the lubricating device is employed within a sheathing assembly for covering the endoscopic tool during its withdrawal from a body passage. Construction of the sheathing assembly will not be described here in detail, since it can be found in our previously mentioned co-pending patent application U.S. Ser. No. 60/641,638 “Improved Surgical Tool For Endoscope”.

The sheathing assembly holds within the housing 80 a flexible sleeve 82, which is retained in an initially bunched state around a guiding tube 84. The guiding tube is intended for guiding the surgical tool along the sheathing assembly when it is axially displaced either distally or proximally. In accordance with the present invention, the guiding tube is filled up with a lubricant 86 and its opposite ends are sealed by respective sealing foils 88, 90. The foils are made of material, which the surgical tool can easily pierce when it enters the housing and is protracted distally out. It can be readily appreciated that in this embodiment the guiding tube functions as the retaining bushing and provides for lubricating the surgical tool.

The present invention is particularly suited for lubricating of tools intended for use with flexible endoscopes. Such endoscopes usually are intended for insertion into the gastrointestinal tract, such as colonoscopes and gastroscopes. It should be kept in mind, however, that the present invention is not limited to flexible endoscopes and is suitable for lubricating surgical tools intended for use with rigid endoscopes as well.

Alternatively, the principles of the present invention may be applied to lubricate tools that are inserted through lumens in medical probes of other types, such as endoscopes (both rigid and flexible) and catheters used in other body passages and in other therapeutic and diagnostic procedures.

It will thus be appreciated that the embodiments described above are cited by way of example, and that the present invention is not limited to what has been particularly shown and described hereinabove. Rather, the scope of the present invention includes both combinations and sub combinations of the various features described hereinabove, as well as variations and modifications thereof which would occur to persons skilled in the art upon reading the foregoing description and which are not disclosed in the prior art.

We claim:

1. A device for lubricating an endoscopic tool comprising
a housing;
a retaining bushing for receiving the surgical tool thereinto and accommodated in said housing, said retaining bushing being provided with a longitudinal through going bore extending between an entry opening and an exit opening;
a lubricating material filling said bore; and,
an entry and an exit closure covering respectively said entry and said exit openings, said entry and exit closures being piercable by the endoscopic tool during insertion thereof in the bushing through the entry opening and protracting thereof distally along the bore.

2. The device for lubricating as defined in claim 1, wherein said entry closure lubricates a shaft of the endoscopic tool during protracting the tool in a distal direction from the entry opening to the exit opening and said exit closure wipes the lubricating material off from the shaft during retracting the tool in a proximal direction from the exit opening to the entry opening.

3. The device for lubricating as defined in claim 2, wherein said closure comprises a plastic foil.

4. The device for lubricating as defined in claim 3, wherein said foil has a thickness of about 5-10 micron.

5. The device for lubricating as defined in claim 4, further comprising a first sealing ring provided between the entry opening and the entry closure and a second sealing ring provided between the exit opening and the exit closure.

6. The device for lubricating as defined in claim 1, wherein said lubricating material is selected from the group comprising paraffin, silicon oil, glycerin, water solution of a resin, water solution of polyethylene oxide and vegetable oil.

7. The device for lubricating as defined in claim 5, wherein said sealing rings being provided with a conical aperture, which is dimensioned and configured to fit tightly around the shaft of the surgical tool.

8. The device for lubricating as defined in claim 7, wherein the conical aperture narrows in the distal direction.

9. The device for lubricating as defined in claim 5, wherein said sealing rings being retained in place by a snapping engagement between an annular protrusion made on an inwardly facing surface of the housing and an annular recess made on a circumference of the sealing ring.

10. The device for lubricating as defined in claim 1, further comprising an adaptor coupled with a distal end of the housing, said adapter securing the device on a port of an endoscope.

11. The device for lubricating as defined in claim 2, wherein at least one of said entry closure and said exit closure is configured and dimensioned to seal the tool shaft when the tool is advanced in the distal direction.

12. The device for lubricating as defined in claim in claim 10, wherein said closures being provided with a variable thickness and said adapter is provided with a lower portion having a conical aperture, which narrows in the proximal direction.

13. The device for lubricating as defined in claim 1, wherein said device is disposable.

14. A combination of an endoscopic tool and a device for lubricating a shaft of the endoscopic tool, said device comprising a housing, which accommodates therein a retaining bushing for receiving the surgical tool thereinto, said retaining bushing is provided with a longitudinal through going bore extending between an entry opening and an exit opening, wherein said bore is filled up with a lubricating material and said entry opening and said exit opening is covered by a respective entry and exit closure, which is piercable by the endoscopic tool during insertion thereof in the bushing through the entry opening and protracting thereof distally, wherein at least one of the said entry closure and the exit closure is adapted to fit tightly around the shaft of the endoscopic tool after the closure has been pierced.

15. The combination as defined in claim 14, further comprising an adaptor coupled with a distal end of the housing, said adapter is designed for securing the combination on a port of an endoscope.

16. The combination as defined in claim 15, further comprising a cover fitted on said device for lubricating, said cover being detachably connectable to the adapter, and said cover beingconfigured and dimensioned to protect a working end of the endoscopic tool from contamination or damage.

17. A method for lubricating of a shaft of an endoscopic tool, said method comprises:
providing a receptacle having a longitudinal through going bore extending between an entry opening and an exit opening, said bore being filled up with a lubricating material and said entry and said exit opening being covered by a respective entry closure and exit closure,
bringing the endoscopic tool near to the entry closure,
pushing the endoscopic tool towards the entry closure and piercing the entry closure,
entering the endoscopic tool in the receptacle through the entry closure,
pushing the endoscopic tool through the lubricating material towards the exit closure,
piercing the exit closure and advancing the endoscopic tool further until it protrudes from the receptacle.

18. The method for lubricating as defined in claim 17, wherein at least one of said entry closure and said exit closure provides sealing between respective closure and respective opening.

19. The method for lubricating as defined in claim 18, wherein at least one of said entry closure and said exit closure comprises a plastic foil.

20. The method for lubricating as defined in claim 17, wherein said lubricating material is selected from the group comprising paraffin, silicon oil, glycerin, water solution of a resin, water solution of polyethylene oxide and vegetable oil.
